(19) United States
(12) Patent Application Publication
(10) Pub. No.: US 2010/0112054 A1
(43) Pub. Date: May 6, 2010

(54) TABLETS AND DISCS WITH COMPARTMENTS WITH TWO OR MORE DRUGS FOR RELEASE AT CERTAIN INTERVALS AND WITH SPECIFIC RATES

(75) Inventors: Nicholas A. Peppas, Austin, TX (US); Lisa Peppas, Austin, TX (US)

Correspondence Address: CHALKER FLORES, LLP, 2711 LBJ FRWY, Suite 1036, DALLAS, TX, 75234, US

(73) Assignee: APPIAN LABS, LLC, Austin, TX (US)

(21) Appl. No.: 12/604,564

(22) Filed: Oct. 23, 2009

Related U.S. Application Data
(60) Provisional application No. 61/107,980, filed on Oct. 23, 2008.
(60) Provisional application No. 61/147,577, filed on Jan. 27, 2009.

(51) Int. Cl. A61K 9/24; A61K 9/00; A61K 47/38; A61K 31/522; A61K 31/167

(52) U.S. Cl. 424/472; 424/400; 514/781; 514/263.34; 514/629

(57) ABSTRACT

The present invention includes systems, compositions and methods of making a multilayer modular release system, wherein the layers form a stack of active agent release layers, wherein the stack comprises a body and first and second ends and an impermeable coating surrounding the body of the stack, wherein the active agent is only release from the first, second or both the first and second ends of the stack by diffusion.

### CROSS-REFERENCE TO RELATED APPLICATIONS

This application claims priority to U.S. Provisional Application Ser. No. 61/107,980, filed Oct. 23, 2008; and Ser. No. 61/147,577, filed Jan. 27, 2009, the entire contents of each are incorporated herein by reference.

### TECHNICAL FIELD OF THE INVENTION

The present invention relates in general to the field of drug release systems, and more particularly, to systems, compositions and methods for controlling drug release profiles.

### BACKGROUND OF THE INVENTION

Without limiting the scope of the invention, its background is described in connection with composition and methods to prepare a composition with various release profiles.

A major unsolved problem in the drug delivery field is the development of pharmaceutical formulations that can release drugs at prescribed delivery rates over a period of time. Currently, conventional pharmaceutical formulations (tablets, capsules, caplets, etc) do not provide a sufficiently controlled release of a drug over a period of time. As seen from standard pharmaceutical references (e.g., J. Robinson and V H L Lee, Controlled Drug Delivery, second edition, Dekker, New York, 1987), tablets produced by compression of microparticles do not provide conditions for predesigned release profiles of drugs when taken orally, buccally, sublingually, ocularly, rectally or by other conventional routes of administration.

Conventional pharmaceutical formulations are prepared by incorporating various excipients and active agents (e.g., drugs) at different concentrations and compressing the ensuing mixture to achieve useful tablets and related systems. Such formulations are usually triggered by the physiological environment. For example, in oral delivery systems the pharmaceutical formulation is placed in contact with the gastrointestinal (GI) contents such as the presence of water, ions, etc.

Such pharmaceutical formulations and controlled release systems work due to the associated swelling in the administration route, the swelling being provided by physiological liquids naturally available in the mouth (saliva), stomach (gastrointestinal fluid), etc. Formulations for drug delivery are very often prepared as monoliths, tablets or matrices, formed by compression of hydrophilic microparticulate powders. These formulations are typically composed of a drug and a hydrophilic swellable excipient (polymer). Many natural or synthetic polymers such as xanthan gum, guar gum, amylose starches, karaya gum, poly(ethylene oxide) (PEO), poly(vinyl alcohol) (PVA) and others have been used. Various cellulose derivatives such as hydroxypropyl methyl cellulose (HPMCs), hydroxypropyl cellulose (HPCs), carboxymethyl cellulose (CMC) and ethyl cellulose (EC) are certainly the most widely used class of polymers for their manufacture.

One such example can be found in the U.S. Pat. No. 5,780,057, which describes a pharmaceutical form for oral administration including a two- or three-layered tablet, with at least one layer that can rapidly swell by contact with biological and/or aqueous fluids, the swelling results in a considerable increase in the tablet volume. The patent shows a prolonged residence of the pharmaceutical form at the gastric level and therefore allows a slow release of the active ingredient from the pharmaceutical form to the stomach and/or the first tract of the intestine.

Another example can be found in the U.S. Pat. No. 5,681,583, which teaches multilayered controlled-release solid pharmaceutical composition in tablet form suitable for oral administration having at least two layers containing active material in association with excipients and additives. One layer of the tablet releases a portion of the drug quickly while the other layer and optionally further layers release portions of the drug more gradually. The patent also teaches systems for the release of active principles which are capable of releasing active principle(s) into an aqueous medium at a controlled rate. For example, a monolithic system for the controlled release of active principles is that that includes: at least one swelling layer containing one or more active principles, in a matrix of swellable, hydrophilic polymers; at least one erodible and/or soluble layer comprising excipients and/or water soluble polymers, possibly containing one or more active principles, either the same or different from those present in the layer, the erodible and/or soluble layer being in contact with the swelling layer(s).

However, all of the above mentioned references do not posses a simple preparation method for formulating the composition, and most of them cannot provide a predesigned release profile of the active agent. Accordingly, there exists a need in the industry for a pharmaceutical composition having a predesigned release profile, with simple methods of preparing the pharmaceutical composition.

### SUMMARY OF THE INVENTION

The present invention includes tablets of layers that are capable of a wide array of release profiles. For example, a tablet with four or more layers that include an excipient alone or drugs in an excipient, or pure drug, for release at certain intervals and with specific rates. For example, the tablets with the four or more layers can be formulated as an oral release system that has an overall customized drug release profiles where each layer provides different drug concentrations at different levels based upon the drug diffusion through the various layers. The entire table has an impermeable coating on the outside that provides for a cylindrical release mechanism. Thus, the rate of diffusion is related to the concentration and the distance in which it travels.

A multilayer modular release system, composition comprising: a total of four or more agent release layers, where the layers form a stack of active agent release layers, wherein the stack comprises a body and first and second ends; and an impermeable coating surrounding the body of the stack, wherein the active agent is only release from the first, second or both the first and second ends of the stack by diffusion. In one aspect, one end can be coated with an impermeable coating. In one aspect, the at least one of the active agent release layers comprises no active agent and/or one or more inactive agents. In another aspect, the pharmaceutical composition comprises an extended release profile, a pulsatile release profile, a delayed release profile, a rapid release followed by a constant release profile, an increasing release profile or a concurrent release profile.

In yet another embodiment, the present invention includes a multilayer, multiple profile drug release pharmaceutical composition comprising: four or more discs that form a stack of discs, wherein each of the discs comprises an active agent releasing polymer and an active agent having a concentration of between 0 and 99.9% by weight of the disc, wherein the stack of discs comprises a body and first and second ends; and an impermeable coating surrounding the body of the stack, wherein the active agent is only release from the first, second or both the first and second ends of the stack by diffusion. In one aspect, the layers or discs have the same concentration of active agent, have a different concentration of active agent, or have alternating concentrations of active agent. In another aspect, the pharmaceutical composition further comprises one or more layers of an extended-release coating, in yet another aspect, the pharmaceutical composition further comprises one or more inactive agents.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show a side view of one an embodiment of the present invention;

FIG. 2 shows a model drug release profile;

FIG. 3 is a graph of another model drug release profile;

FIGS. 4A to 4E show the structure of the layers and the control over one release profile of the present invention;

FIGS. 5A to 5D show the structure of the layers and the control over the pulsatile release profile of the present invention;

FIGS. 6A and 6B show the structure of the layers and the control over the delayed release profile of the present invention;

FIGS. 7A and 7B show the structure of the layers and the control over the bust, then constant, release profile of the present invention;

FIGS. 8A to 8D show the structure of the layers and the control over the increasing release profile of the present invention; and

FIGS. 9A and 9B show the structure of the layers and the control over the concurrent release profile of the present invention.

### DETAILED DESCRIPTION OF THE INVENTION

While the making and use of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

A number of definitions are provided herein to facilitate an understanding of the present invention. As used herein, the term “enveloped pharmaceutical” refers to a capsule, a suppository, a gel cap, a softgel, a lozenge, a sachet or even a fast dissolving wafer. As used herein the term “carrier” is used to describe a substance, whether biodegradable or not, that is physiologically acceptable for human or animal use and may be pharmacologically active or inactive.

The pharmaceutical composition and/or the solid carrier particles may be coated with one or more enteric coatings, seal coatings, film coatings, barrier coatings, compress coatings, fast disintegrating coatings, or enzyme degradable coatings. Multiple coatings on the ends of the layers or discs may be applied for desired performance. For example, one or more agents that delay release until the proper pH, gel formation and/or timed-release polymers and/or additives are provided. Further, some actives may be provided for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The compositions may be formulated and packaged for, e.g., oral, nasal, buccal, ocular, urethral, transmucosal, vaginal, topical or rectal delivery. Pharmaceutical Glaze, e.g., shellac is a natural occurring material that may be used to coat the sides of the layers or discs to prevent release of any active agent from the side(s) of the delivery system of the present invention. The main component of shellac (˜95%) is a resin that upon mild basic hydrolysis gives a mixture of compounds of high plasticity. Shellac is used extensively in the pharmaceutical industry as a film coating agent for beads and tablets.

The sides of a tablet that includes the active agent release layers can be coated with an impermeable coating or layer during manufacture and/or following final packaging or can be inserted into a sleeve that prevents dispersal of any active agent from the sides of the final tablet, or both. Non-limiting exemplary materials to be used to form the impermeable layer include, e.g., a polymer film (or sheet) capable of allowing a medicine in contact therewith to migrate therethrough is a film (preferably having a thickness of from about 10 to 1,000 μm) of a homopolymer, block copolymer or copolymer that are generally biocompatible and/or biodegradable. Non-limiting illustrative examples of polymers, coatings or films that may be used to cause at least one side of the tablet or pharmaceutical composition to be impermeable to the environment for drug delivery include, polyvinyl acetate, a copolymer of vinyl acetate and a monomer copolymerizable with vinyl acetate, and a polymer containing alkoxy acrylate, polyethylene, polypropylene, polyethylene-oxide, polyvinylidene chloride, polyester, polyamide, cellophane, metal foil, natural or synthetic resins and/or rubbers or combinations thereof. Non-limiting examples of synthetic resins include: polyvinyl alkyl ethers, polyacrylates, polymethacrylates, polyurethanes, polyesters, polyamides, and ethylene-vinyl acetate copolymers. Non-limiting examples of rubbers include: styrene-isoprene-styrene block copolymer rubber, styrene-butadiene rubber, polybutene rubber, polyisoprene rubber, butyl rubber, silicone rubber, and natural rubber.

In one example, the impermeable coating process involves spraying an impermeable coating solution onto a substrate. The coating solution can be a molten solution of the encapsulation coat composition free of a dispersing medium. The coating solution may also be prepared by solubilizing or suspending the composition of the encapsulation coat in an aqueous medium, an organic solvent, a supercritical fluid, or a mixture thereof. At the end of the coating process, the residual dispersing medium can be further removed to a desirable level using appropriate drying processes, such as vacuum evaporation, heating, freeze drying, etc. Depending on the equipment of manufacture, shape of the tablet and the binding surface, the coating may be directionally sprayed such that only the side(s) of the tablet is/are coated. In other examples, the entire tablet may be coated and then one or more ends may be exposed by mechanically (e.g., cutting, shearing, abrading), chemically (dissolving one or more sides), or combinations thereof (e.g., etching, laser cutting, heating or melting). In certain embodiments, the coating on the side that will be exposed to the environment and will permit the release of active agent(s) (i.e., the non-impermeable side or sides) may also be coated temporarily in order to deliver the active agent to a specific location. For example, the temporary coating may be an enteric coating that prevents release of the active agent(s) in the stomach but releases in the intestines. Another example of a temporary coating is an effervescent coating that dissolves upon contact with a solvent that triggers release of the effervescent portion prior to exposing the layers of the present invention to the release environment. Temporary coatings may be formed or deposited onto the tablet before, concurrently or after the formation of the impermeable layer.

To form the tablet, one or more additives may be included in or between the layers, such as binders (adhesives), i.e., agents that impart cohesive properties to powdered materials through particle-particle bonding, such as matrix binders (dry starch, dry sugars), film binders (polyvinylpyrrolidone (PVP), starch paste, celluloses, bentonite and sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose).

There are a number of possible options for coating these tablets selectively along the cylindrical face and possibly one circular face (e.g., pan coating, enrobing, electro-static deposition, plasma coating, spray coating, banding and extrusion technologies). For example, the ingredients may be adjusted at one or both ends such that coating applied uniformly will not adhere to the cylindrical sides of the tablet. Another option is the inclusion of a very fast swelling excipients in one or both ends such that minimal fluid uptake through the coating. Yet another option is to include intentional flaws in the coating that results in rapid swelling and removal of tablet end(s). Depending on the selected method, the coating can be impermeable or semi-permeable.

Swelling tablets and matrix compositions are key systems in the field of drug delivery systems. Water swelling behavior into cross-linked polymers has been demonstrated over past several decades, and it becomes an important factor when considering how such compositions will behave in the GI tract, e.g., under what conditions they will give substantially constant rate release (zero order release) or continuously decreasing release (Korsmeyer, R. W., R. Gurny, E. Doelker, P. Buri, and N. A. Peppas, “Mechanisms of Solute Release from Porous Hydrophilic Polymers,” Intern. J. Pharm., 15 (1983) 25-35). Typically, zero order release is difficult or impossible to achieve with swelling systems. It requires that a carrier (polymer) be mixed with drug, dried for several hours or days, cut in disc shape and used as a composition. This is an unacceptable method for the pharmaceutical industry due to cost and time constrains.

The use of swellable materials for drug delivery applications has followed investigations of solvent and solute transport in polymeric systems, with several important observations and mathematical models developed which describe transport behavior in polymeric systems. For example, polymeric hydrogels have been used for the purpose of extended drug delivery, as well as drug targeting and patterned release profiles (Colombo, P., “Swelling-controlled Release in Hydrogel Matrices for Oral Route,” Adv. Drug Deliv. Rev., 11 (1993) 37-57). Typical pharmaceutical compositions e.g., tablets, include an active ingredient compressed in a powder e.g., a cellulose derivative, and a disintegrant. However, these systems can only control drug release during the initial stages after the drug is placed in a body. Coated capsules can protect a drug for a period of time before releasing at an optimal site, thereby prolonging the active lifetime of the drug. These systems have limited applications for long-term drug delivery. Hydrogel delivery systems are capable of slow release of an imbedded drug, with release controlled by the rate of swelling and relaxation of the polymer (Klier, J. and N. A. Peppas, “Solute and Penetrant Diffusion in Swellable Polymers. VIII. Influence of the Swelling Interface Number on Solute Concentration Profiles and Release,” J. Control. Rel., 7 (1988) 61-68. Peppas, N. A. and A. R. Khare, “Preparation, Structure and Diffusional Behavior of Hydrogels in Controlled Release,” Adv. Drug Deliv. Rev., 11 (1993) 1-35).

Other examples, such as drug/solute release from pH-sensitive materials, can have additional factors influencing release profiles. Brannon-Peppas, L. and N. A. Peppas, “Solute and Penetrant Diffusion in Swellable Polymers. IX. The Mechanisms of Drug Release from pH-Sensitive Swelling-Controlled Systems,” J. Control. Rel., 8 (1989) 267-274) demonstrated swelling and release behavior of pH-sensitive hydrogels.

Excipients for use with the present invention:

| | Common Range | Maximum Range |
|---|---|---|
| Excipient | (wt %) | (wt %) |
| HPMC 15 | 9.99-78.32 | 5-80 |
| HPMC 100 | 42.42 | 40-70 |
| Guar Gum | 9.95-10.47 | 2-15 |
| Sodium CMC | 4.99-5.23 | 2-10 |
| Talc | 0.102-0.112 | 0.05-0.25 |
| Magnesium Stearate | 0.201-0.211 | 0.1-0.5 |
| Avicel (MCC:mannitol blend) | 37.87 | 20-50 |
| Vivasol (Soldium Starch | 2.01 | 1-5 |
| Glycolate) | | |

AppiForm™ Modular Release System.

The present invention includes tablets of layers that are capable of a wide array of release profiles. For example, a tablet with four or more layers (each layer may include an excipient alone or drugs for release at certain intervals) with specific rates of drug release. The present invention includes an oral release system that has customized drug release profiles. In one example, the invention includes more than four layers, wherein each of the layers can provide different concentrations, different thicknesses for the layers or provide different diffusion rates at each later. An impermeable coating on the outside provides for a cylindrical release mechanism. Thus the rate of diffusion is related to the concentration and the distance that the drug travels.

The present invention can be made with standard tablet pressing equipment and techniques to make multiple layers into a flat or cylindrical tablet in which the impermeable sides allow for the outer layers (at one or both ends) to provide a release profile and then the inner layers (that take longer to diffuse through the ‘matrix’) would also have a desired release profile. This is not dissolution, but rather, diffusion over various distances. Importantly, there is no dependency on pH, fed or fasted state, etc. that might increase or decrease standard diffusion, that can be used to mathematically calculate/customize release profiles. In the system of the present invention, the release is dependent upon all the layers—it is not a simple bi-layer table or two tablets of differing rates stuck together.

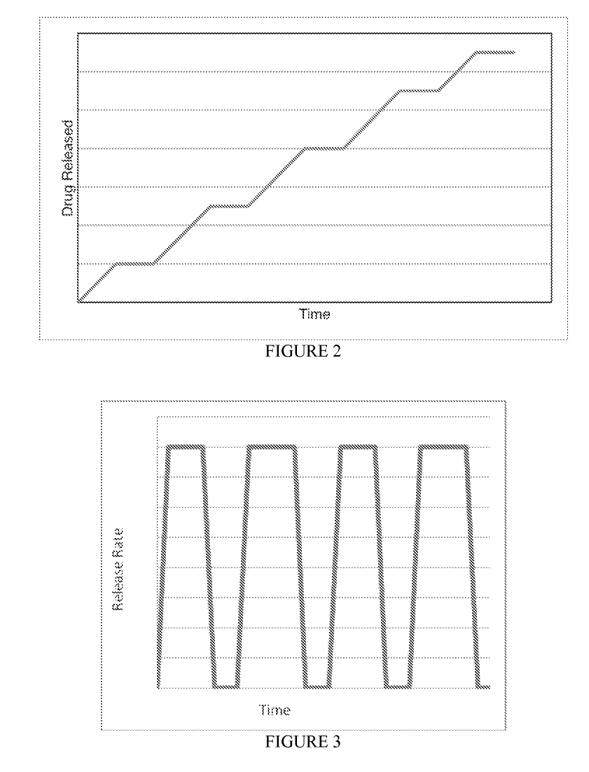

The present invention addresses this phenomenon and process, and especially the ability to prepare well controlled systems with predesigned intervals. Delivery of therapeutic agents for treatment of a number of diseases requires the ability to release more than on drugs either sequentially or in other modes of release such as intermittent delivery of a drug with well-controlled intervals. General patterns of drug delivery as those shown in FIGS. 2 and 3 are not easy to achieve as they require that several sections, areas or layers of the pharmaceutical device function at precise intervals.

In the invention described herein, a pharmaceutical tablet of, e.g., 5, 10, 15, 50, 100, 200, 250, 300, 400, 500, 750, 1,000 or more milligram total weight is prepared by compressing several layers of pharmaceutical excipients in one or more configurations that achieve the desirable effect.

Intermittent Release of One Drug with Initial Drug Delivery.

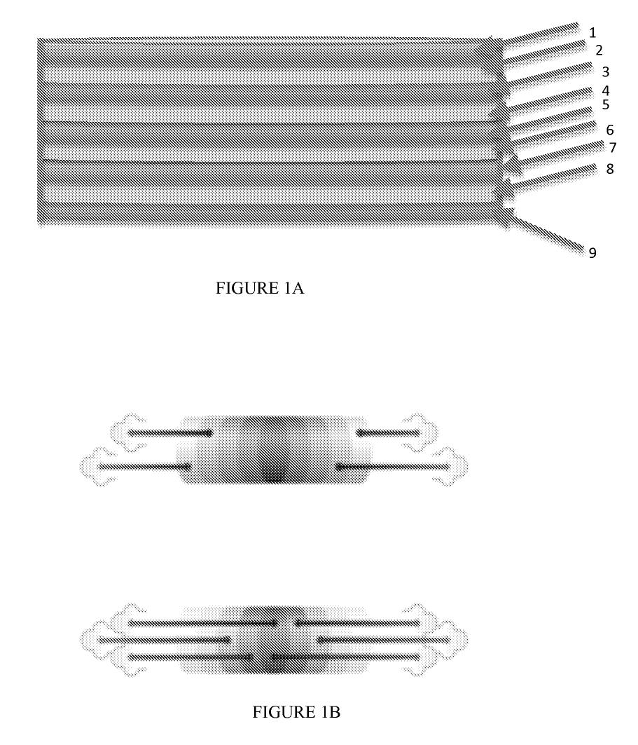

The tablet depicted in FIG. 1A includes, e.g., 5, 7 or 9 layers (although more or fewer layers can be used) where the layers include a drug mixed or dispersed in a hydrophilic excipient, such as, hydroxypropylmethyl cellulose (HPMC), carboxy methyl cellulose (CMC) and related cellulose-based or other excipients. The tablet may include 4 or more layers. The layers depicted are not indicative of whether the layer has a drug or not, but are present solely to distinguish between the layers. The preferred composition of the layers is between 1:99 and 70:30 weight by weight ratio of the granulated dug to the polymer and excipient. Small amounts of a binder and lubricant are added as needed.

The layers may include a pure polymer excipient, e.g., a mixture of one or more hydrophilic excipients such as, e.g., hydroxypropylmethyl cellulose (HPMC) or hydroxypropyl cellulose (HPC) and/or with, e.g., a hydrophobic excipient ethyl cellulose (EC) in weight ratios of 5:95 and up to 60:40. The center layer (layer 5 in FIG. 1A) may have a composition such as the odd-numbered layers or may be pure drug. FIG. 1B shows one the left side the variable concentration of drug in the tablet, and on the right side the locations of the release of the drug from the dosage form, depicted as being released from the ends of the tablet because the cylindrical sides of the layers of the tablet are coated to prevent diffusion of the drug from the sides of the tablet.

Intermittent Release of One Drug with Initial Delayed Action.

The tablet may include 4, 5, 6, 7, 8 or 9 layers (although more layers can be added) where, e.g., the odd-numbered layers, are made from a pure polymer excipient, e.g., a mixture of a hydrophilic excipient such as hydroxypropylmethyl cellulose (HPMC) or hydroxypropyl cellulose (HPC) with a hydrophobic excipient ethyl cellulose (EC) in weight ratios of 5:95 and up to 60:40.

The even-number layers comprise a drug to be released, granulated and then mixed or dispersed in a hydrophilic excipient such as a hydroxypropylmethyl cellulose (HPMC), carboxy methyl cellulose (CMC) and related ones. The preferred composition of the odd-numbered layers is between 1:99 and 70:30 weight by weight ratio of the granulated dug to the polymer excipient. Small amounts of a binder and lubricant are added as needed.

The center layer (layer 5 in FIG. 1) may be pure excipient or may have a composition such as the odd-numbered layers or may be pure drug.

Release Behavior of One Drug with Immediate Release.

The drug release behavior from the tablet is described in FIG. 2. The drug release for the first formulation is intermittent, and increasing in amount as time passes. The associated drug release rate is shown in FIG. 3.

Control of the Duration Release Intervals.

The duration of the releasing portions of the curves is controlled by the composition of the drug-containing layers and the thickness of said layers.

The study of the release of a drug over the time is crucial information in the drug delivery field. The usage of mathematical models when designing new pharmaceutical formulations as well as for the analysis of the experimental results is essential. Studies of the drug delivery profile are based on basic diffusion equations. The release of a drug from a swollen layer or area of a hydrogel involves the movement of the drug molecules through the bulk of the polymer. This phenomenon is known as diffusion and can be explained by mass transport fundamentals. From a macroscopic point of view, Fick's laws of diffusion can explain the movement of drug molecules from the hydrogel matrix to the external environment. Equation (1) presents Fick's second law of diffusion. This is an one-dimensional form of Fick's law.

$$\frac{\partial C}{\partial t} = D ( \frac{{\partial }^{2} C}{\partial {x}^{2}} ) \qquad ( 1 )$$

In these equations, the concentration and mass flux are designated as C and J, respectively. D is the diffusion coefficient. The independent variables of position and time are designated as x and t, respectively.

In monolithic devices the drug is intimately mixed with the polymer which has rate-controlling properties. The drug can then be dissolved or dispersed in the polymer, resulting in two different types of monolithic systems. Therapeutic agents released from monolithic delivery systems do not follow zero order kinetics, although they provide a prolonged drug release.

As indicated above there will be times when the center compartment will be pure drug. These are then reservoir devices where the drug is contained in a core which is surrounded by a rate-controlling polymeric membrane. Drug transport from the core through the external polymer membrane occurs by dissolution at one interface of the membrane and diffusion driven by a gradient in thermodynamic activity.

From these equations, it is observable that drug release can be controlled by the thickness of the polymer excipient, the concentration difference of the drug across the polymer, the thermodynamic characteristics of the systems through the partition coefficient and the structure of the polymer via the drug diffusion coefficient. Therefore, the drug release can be modeled.

Release of Two or More Drugs.

This technology allows for the release of multiple layers of drug, or a drug and a masking agent, etc. In a general method, certain layers are loaded with the beneficial agent that is going to be released, while other layers are filled with another agent or a masking agent.

Varied Release.

This technology allows for the release of multiple layers of drug, or a drug and a masking agent, etc. In a general method, certain layers are loaded with the beneficial agent that is going to be released, while other layers are filled with another agent or a masking agent such that the release profiled may be varied.

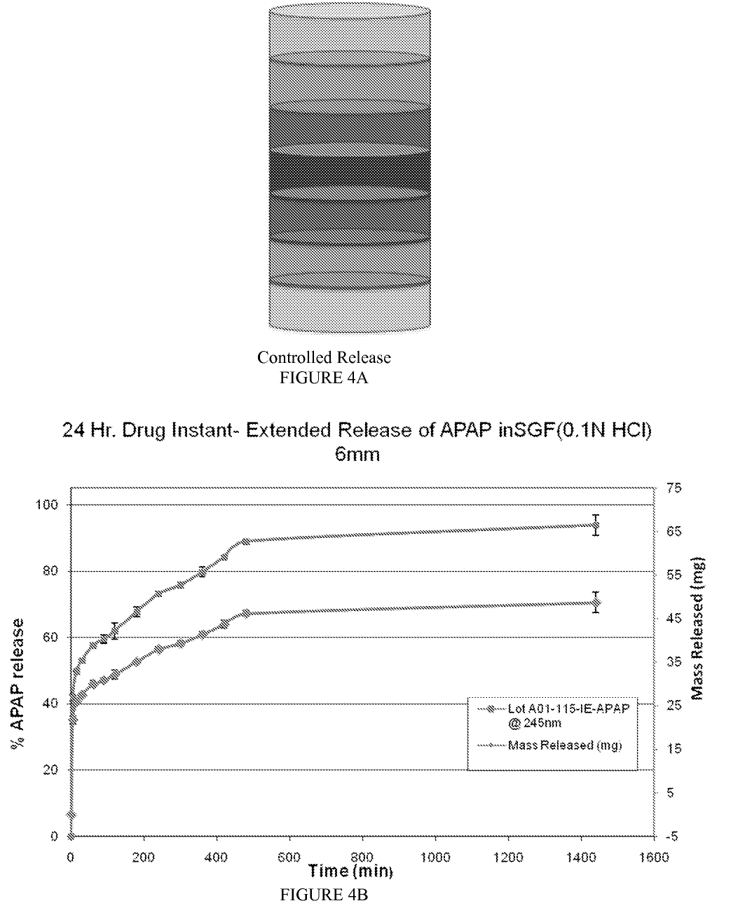

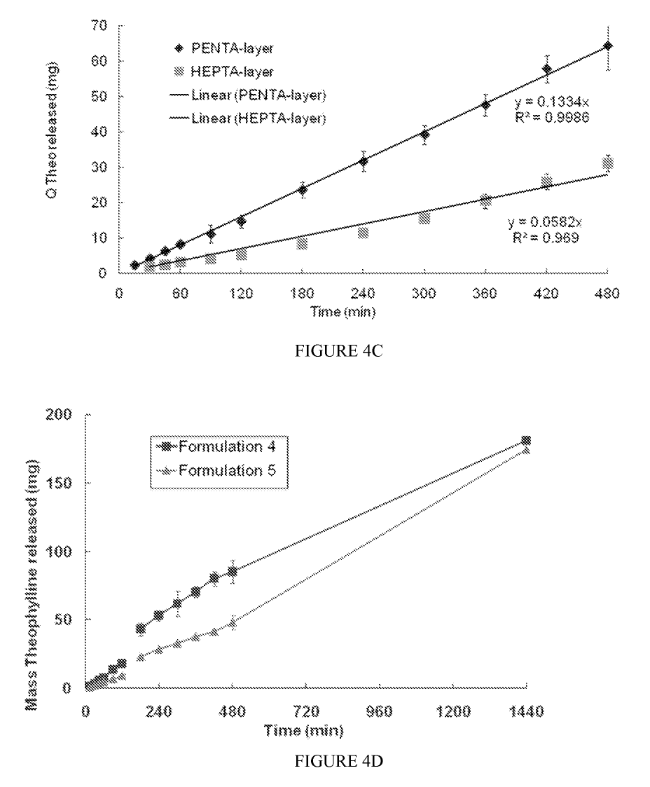

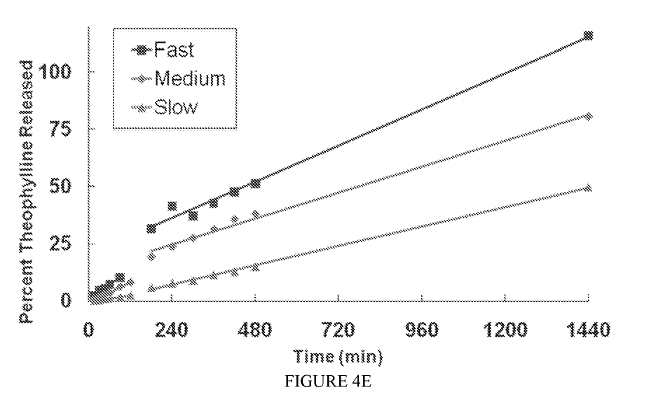

FIGS. 4A to 4E show the structure of the layers and the control over the release profile of the present invention.

Pulsatile Drug Release.

Pulsatile Drug Release was achieved by forming tablets using direct compression in following manner. Each layer of this tablet was 100 mg, the first layer of the tablet was comprised of 25% theophylline and a 75% excipient blend (60% Methocel K15M, 10% Guar gum, 5% Sodium CMC, and 0.1% talc) it was geometrically diluted, added to top of first anvil and pressed in 13 mm die under 500 psi for 1 second. The next layer working up was 0% theophylline and 100% excipient blend (85% Methocel K15M, 10% Guar gum, 5% Sodium CMC, and 0.1% talc), it was geometrically diluted, added on top of previous layer and pressed in 13 mm die under 500 psi for 1 second. The third layer was 50% theophylline and 50% excipient blend (35% Methocel K15M, 10% Guar gum, 5% Sodium CMC, and 0.1% talc) it was geometrically diluted, added on top of previous layer and pressed in 13 mm die under 500 psi for 1 second. The fourth layer of the gradient was 0% theophylline and 100% excipient blend (85% Methocel K15M, 10% Guar gum, 5% Sodium CMC, and 0.1% talc) it was geometrically diluted, added on top of previous layer and pressed in 13 mm die under 500 psi (3,447 Kpascals) for 1 second. The sixth layer of the gradient was 0% theophylline and 100% excipient blend (85% Methocel K15M, 10% Guar gum, 5% Sodium CMC, and 0.1% talc) it was geometrically diluted, added on top of previous layer and pressed in 13 mm die under 500 psi for 1 second. The seventh layer of the gradient was 50% theophylline and 50% excipient blend (35% Methocel K15M, 10% Guar gum, 5% Sodium CMC, and 0.1% talc) it was geometrically diluted, added on top of previous layer and pressed in 13 mm die under 500 psi for 1 second. The eighth layer of the gradient was 0% theophylline and 100% excipient blend (85% Methocel K15M, 10% Guar gum, 5% Sodium CMC, and 0.1% talc) it was geometrically diluted, added on top of previous layer and pressed in 13 mm die under 500 psi for 1 second. The ninth and final layer of the gradient was 25% theophylline and 75% excipient blend (60% Methocel K15M, 10% Guar gum, 5% Sodium CMC, and 0.1% talc) it was geometrically diluted, added on top of previous layer and pressed in 13 mm die under 500 psi for 1 second, then the second anvil was added to the top layer and a final pressing of the tablet was performed under 2500 psi for 5 seconds, in order to lock ingredients into place. The lateral wall of the layered tablet was then coated with an acid/base insoluble polymer in order to create an impermeable membrane around the tablet to divert the diffusion of actives down their concentration gradient. The layers can then be inserted into a sleeve that prevents dispersal of any active agent from the sides of the final tablet, or the sides of the tablet can be coated with an impermeable coating or layer during manufacture and/or following final packaging.

Dissolution testing of these tablets was performed using a dissolution apparatus with 900 ml simulated gastric fluid (USP buffers, solns and media) as media @37° C. with a paddle speed of 50 rpms. Time points were taken at 15 min., 30 min., 60 min., 90 min., 2 Hrs., 3 Hrs., 4 Hrs., 5 Hrs., 6 Hrs., 7 Hrs., 8 Hrs., and 24 Hrs. to demonstrate the full drug release profile in time. Samples filtered through a 0.45 um PTFE syringe filter prior to analysis. UV-Vis absorbance analysis of the dissolution samples was performed using a Gen-5 UV-Vis spectrophotometer. Sample absorbance was measured at 245 nm, compared a stock standard calibration curve.

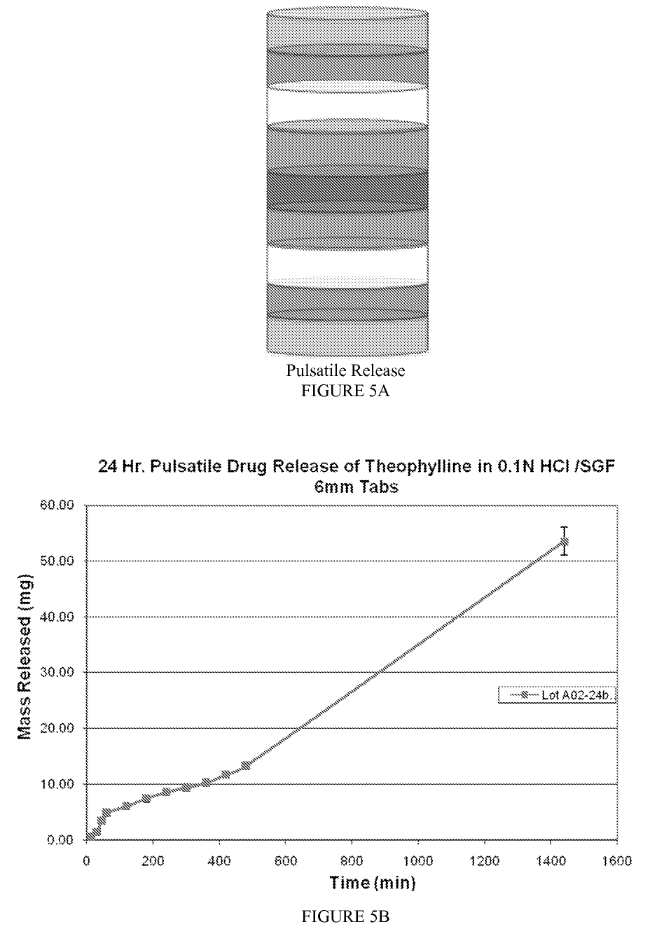

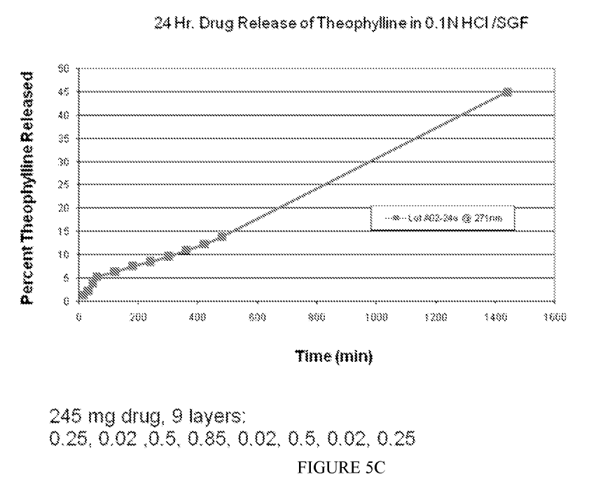

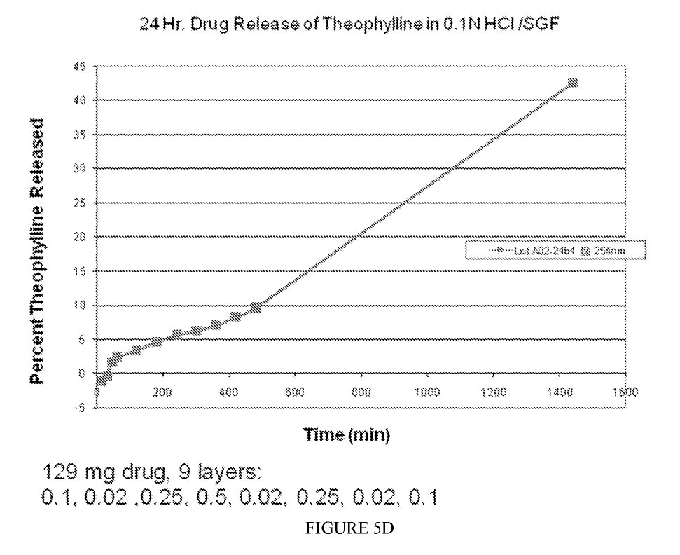

FIGS. 5A to 5D show an example of the structure of the typical layers and the control over the pulsatile release profile of the present invention.

Delayed Release

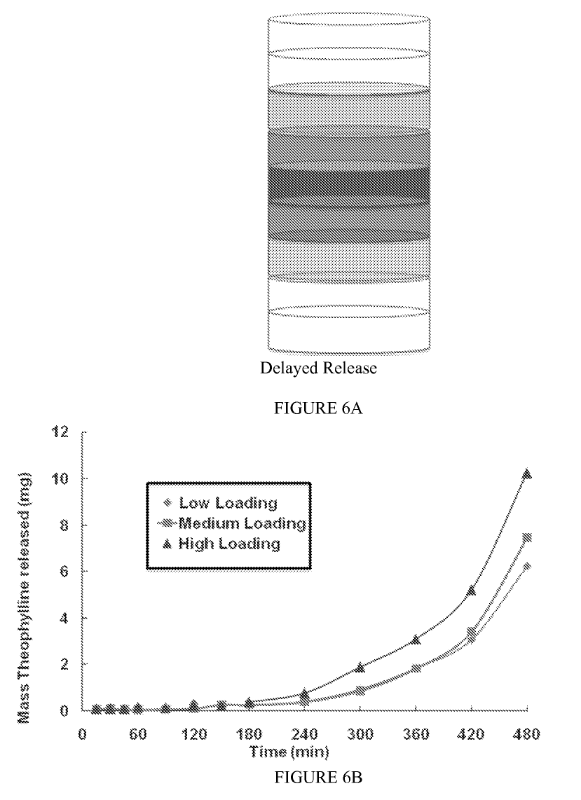

FIGS. 6A to 6D show an example of the structure of the typical layers and the control over the delayed release profile of the present invention.

Rapid then Constant or Rapid/Extended Drug Release.

Rapid/Extended Drug Release was achieved by forming tablets using direct compression in following manner. Each layer of this tablet was 15 mg, the first layer of the tablet consisted of 60% acetaminophen and a 40% excipient blend (37.9% HFE-10Z, 2% vivasol and 0.1% talc) it was geometrically diluted added on top of the first anvil and pressed in 6 mm die under 500 psi for 1 second. The next layer working up was 25% acetaminophen and 75% excipient blend (59% Methocel K15M, 5% Sodium CMC, 10% Guar Gum and 0.1% talc), it was geometrically diluted, added on top of previous layer and pressed in 6 mm die under 500 psi for 1 second. The third layer was 50% acetaminophen and 50% excipient blend (34% methocel K15M, 5% Sodium CMC, 10% guar gum, and 0.1% talc) it was geometrically diluted, added on top of previous layer and pressed in 6 mm die under 500 psi for 1 second. Finally the last layer of the gradient was 75% acetaminophen and 25% excipient blend (10% methocel, 5% Sodium CMC, 10% guar gum, and 0.1% talc) it was geometrically diluted, added on top of previous layer and pressed in 6 mm die under 500 psi for 1 second. The final outer layer of the tablet is identical to the first. This layer was added to the previous layer and pressed under 500 psi for 1 second, then the second anvil was added to the top layer and a final pressing of the tablet was performed under 2500 psi for 5 seconds, in order to lock ingredients into place. The lateral wall of the layered tablet was then coated with an acid/base insoluble polymer in order to create an impermeable membrane around the tablet to divert the diffusion of actives down their concentration gradient.

Dissolution testing of these tablets was performed using a dissolution apparatus with 900 ml simulated gastric fluid (USP buffers, solutions and media) as media at 37° C. with a paddle speed of 50 rpms. Time points were taken at 15 min., 30 min., 60 min., 90 min, 2 Hrs., 3 Hrs., 4 Hrs., 5 Hrs., 6 Hrs., 7 Hrs., 8 Hrs., and 24 Hrs. to demonstrate the full drug release profile in time. Samples filtered through a 0.45 um PTFE syringe filter prior to analysis. UV-Vis absorbance analysis of the dissolution samples was performed using a Gen-5 UV-Vis spectrophotometer. Sample absorbance was measured at 245 nm and compared to a stock standard calibration curve.

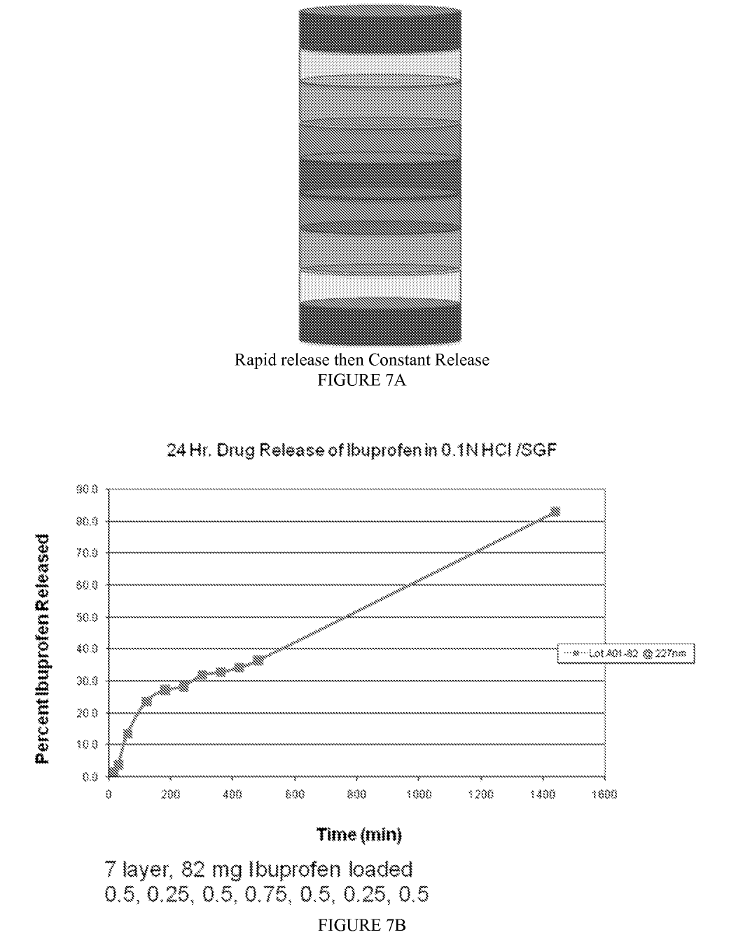

FIGS. 7A to 7B show an example of the structure of the typical layers and the control over the rapid, then constant, release profile of the present invention.

Customized Release-Increasing Rate Drug Release or Increasing Release.

A customized increasing rate drug release was created by forming tablets using direct compression in the following manner. Each layer of this tablet was 25 mg, the first layer of the tablet consisted of 5% theophylline and a 95% excipient blend (80% Methocel K15M, 10% Guar gum, 5% Sodium CMC, and 0.1% talc) it was geometrically diluted, added to the top of first anvil and pressed in a 6 mm die under 500 psi for 1 second. The next layer working up was 10% theophylline and 90% excipient blend (75% Methocel K15M, 10% Guar gum, 5% Sodium CMC, and 0.1% talc), it was geometrically diluted, added on top of previous layer and pressed in 6 mm die under 500 psi for 1 second. The third layer was 85% theophylline and 15% excipient blend 10% Guar gum, 5% Sodium CMC, and 0.1% talc) it was geometrically diluted, added on top of previous layer and pressed in 6 mm die under 500 psi for 1 second. The fourth layer of the gradient was 10% theophylline and 90% excipient blend (75% Methocel K15M, 10% Guar gum, 5% Sodium CMC, and 0.1% talc) it was geometrically diluted, added on top of previous layer and pressed in 6 mm die under 500 psi for 1 second. The fifth and final layer of the tablet was 5% theophylline and 95% excipient blend (80% Methocel K15M, 10% Guar gum, 5% Sodium CMC, and 0.1% talc) it was geometrically diluted, added on top of previous layer and pressed in 6 mm die under 500 psi for 1 second, then the second anvil was added to the top layer and a final pressing of the tablet was performed under 2500 psi for 5 seconds, in order to lock ingredients into place. The lateral wall of the layered tablet was then coated with an acid/base insoluble polymer in order to create an impermeable membrane around the tablet to divert the diffusion of actives down their concentration gradient.

Dissolution testing of these tablets was performed using a dissolution apparatus with 900 ml simulated gastric fluid (USP buffers, solutions and media) as media at 37° C. with a paddle speed of 50 rpms. Time points were taken at 15 min., 30 min., 60 min., 90 min, 2 Hrs., 3 Hrs., 4 Hrs., 5 Hrs., 6 Hrs., 7 Hrs., 8 Hrs., and 24 Hrs. to demonstrate the full drug release profile in time. Samples filtered through a 0.45 um PTFE syringe filter prior to analysis. UV-Vis absorbance analysis of the dissolution samples was performed using a Gen-5 UV-Vis spectrophotometer. Sample absorbance was measured at 245 nm, compared a stock standard calibration curve.

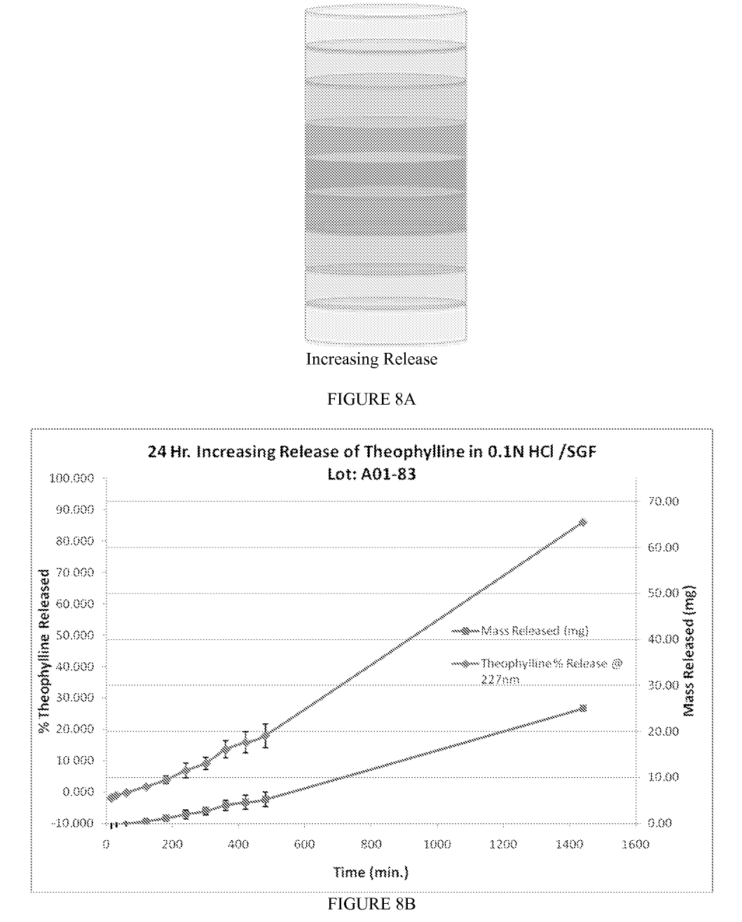

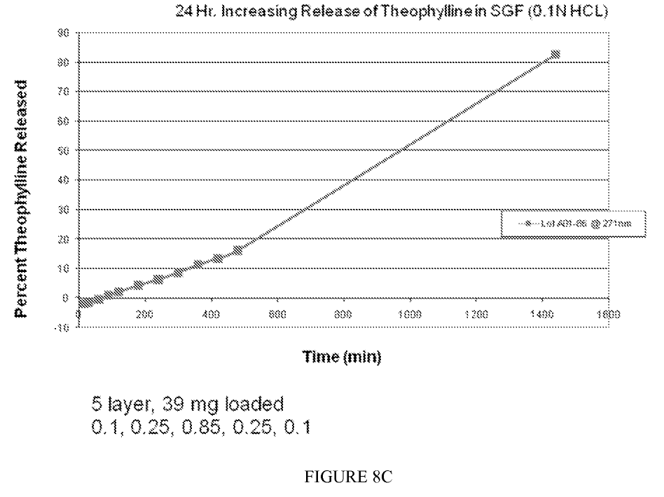

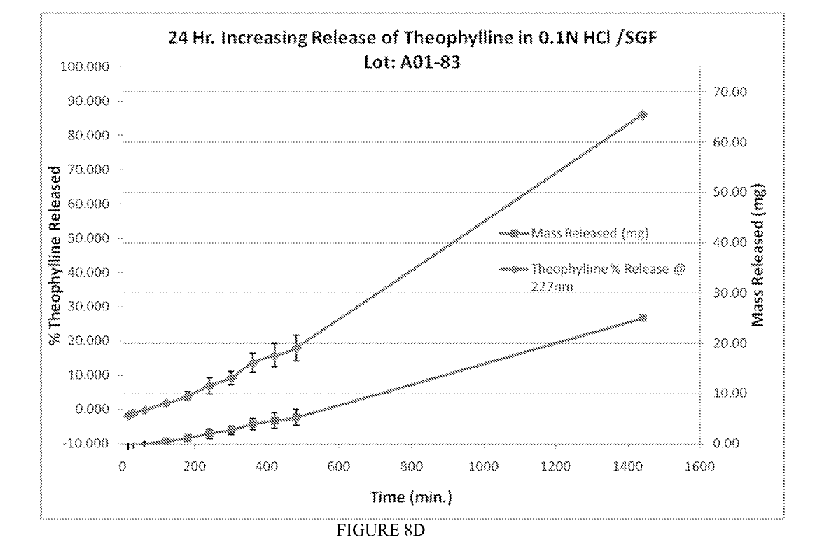

FIGS. 8A to 8B show an example of the structure of the typical layers and the control over the pulsatile release profile of the present invention.

Concurrent Release.

The concurrent drug release system for two active compounds of variable strengths was achieved by using direct compression as a method for forming tablet layers and stacking layers in a concentration. The first layer of the tablet consisted of 20 mg of 25% theophylline and a 75% excipient blend (60% Methocel K15M, 10% Guar gum, 5% Sodium CMC, and 0.1% talc) it was geometrically diluted added on top of the first anvil and pressed in 6 mm die under 500 psi for 1 second. The next layer working up had a mass of 20 mg and was composed of 50% theophylline and 50% excipient blend (35% Methocel K15M, 5% Sodium CMC, 10% Guar Gum and 0.1% talc), it was geometrically diluted, added on top of previous layer and pressed in 6 mm die under 500 psi for 1 second. The third layer had a mass of 20 mg, and was composed of 75% theophylline and 25% excipient blend (10% Methocel K15M, 5% Sodium CMC, 10% guar gum, and 0.1% talc) it was geometrically diluted, added on top of previous layer and pressed in 6 mm die under 500 psi for 1 second. Finally the last layer of the tablet was 60 mg, which contained the second “active ingredient” was 10% Yellow lake and 90% excipient blend (76.5% Methocel K15M, 4.5% Sodium CMC, 9% guar gum, and 0.1% talc) it was geometrically diluted, added on top of previous layer and pressed in 6 mm die under 500 psi for 1 second, then the second anvil was added to the top layer and a final pressing of the tablet was performed under 2500 psi for 5 seconds, in order to lock ingredients into place. The lateral wall of the layered tablet was then coated with an acid/base insoluble polymer in order to create an impermeable membrane around the tablet to divert the diffusion of actives down their concentration gradient.

Dissolution testing of these tablets was performed using a dissolution apparatus with 900 ml simulated gastric fluid (USP buffers, solutions and media) as media at 37° C. with a paddle speed of 50 rpms. Time points were taken at 15 min., 30 min., 60 min., 90 min, 2 Hrs., 3 Hrs., 4 Hrs., 5 Hrs., 6 Hrs., 7 Hrs., 8 Hrs., and 24 Hrs. to demonstrate the full drug release profile in time. Samples filtered through a 0.45 um PTFE syringe filter prior to analysis. UV-Vis absorbance analysis of the dissolution samples was performed using a Gen-5 UV-Vis spectrophotometer. Sample absorbance was measured at 245 nm, compared a stock standard calibration curve.

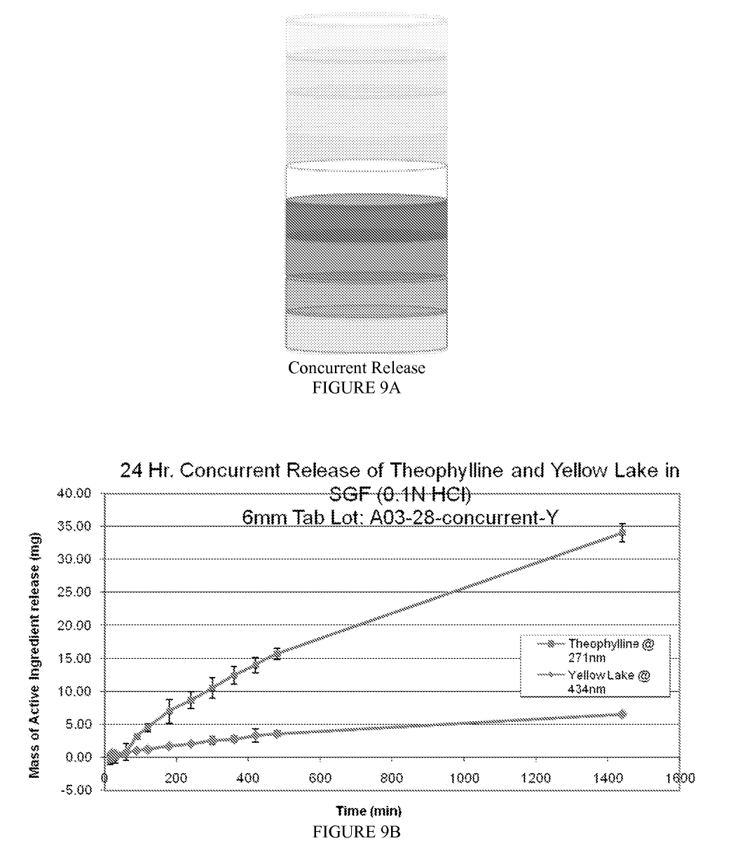

FIGS. 9A to 9B show an example of the structure of the typical layers and the control over the concurrent release profile of the present invention.

As used herein, the terms “layer” and “disc” are used to describe a unit of the overall tablet or compression, which may include 1 to 100 layers or discs, e.g., 1, 2, 3, 4, 5, 6, 7, 8, 9, 10, 20, 30, 40, 40, 50, 60, 70, 80, 90, 100.

As used herein, the terms “extended release” and “delayed release” as used herein are used to define a release profile to effect delivery of an active over an extended period of time. Extended release as used herein may also be defined as making the active ingredient available to the patient or subject regardless of uptake, as some actives may never be absorbed by the animal. Various extended release dosage forms may be designed readily by one of skill in art as disclosed herein to achieve delivery to both the small and large intestines, to only the small intestine, or to only the large intestine, depending upon the choice of coating materials, coating thickness and/or number of different layers with different polymer to drug ratio, and/or different manufacture process.

“Extended release” and “delayed release” compositions may be prepared and delivered so that release is accomplished at some generally predictable location in the lower intestinal tract more distal to that which would have been accomplished if there had been no delayed release alterations. A method for delay of release is, e.g., a coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers and compatible mixtures thereof may be used to provide the coating for the delayed or the extended release of active ingredients, and some of their properties, include, but are not limited to: shellac, also called purified lac, a refined product obtained from the resinous secretion of an insect.

The term “enteric coating” as used herein relates to a mixture of pharmaceutically acceptable excipients that is applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; easy application as a coating (substrate friendly); and economical practicality.

Colorants, detackifiers, surfactants, antifoaming agents, lubricants, stabilizers such as hydroxy propyl cellulose or methylated cellulose, acid/base can be added to the coatings of the present invention besides plasticizers to solubilize or disperse the coating material, and to improve coating performance and the coated product.

The pharmaceutically active agents useful in the practice of the present invention include, but are not limited to, nutraceuticals, vitamins, food additives, food supplements, antihistamines, decongestants, antitussives and/or expectorants. Other actives for use with the present invention include, but are not limited to: non-steroidal anti-inflammatory drugs (NSAIDs) and other analgesic drugs such as acetominophen and phenacetin. These materials are incorporated into the slow or controlled release compositions of the invention in amounts governed by the desired release characteristics of the material in such excipient base and such that conventional dosages comply with applicable FDA or other regulations.

Suitable excipients (active agents) are those used commonly to facilitate the processes involving the preparation of the solid carrier, the encapsulation coating, or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. The excipients may also be pre-coated or encapsulated, as are well known in the art.

Carriers: The carrier of the pharmaceutical compositions may be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A carrier may be a finely divided (milled, micronized, nanosized, precipitated) form of a matrix on which the active ingredient is disposed. Such matrix may be formed of various materials known in the art, such as, for example: sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapatite, tricalcium phosphate, talc, or titania; and polyols, such as mannitol, xylitol, sorbitol or cyclodextrin. It should be emphasized that a substrate need not be a solid material, although often it will be a solid.

Other additives conventionally used in pharmaceutical compositions may be included in the present invention, which are known in the art. Such additives include, e.g., anti-adherents (anti-sticking agents, glidants, flow promoters, lubricants) such as talc, magnesium stearate, fumed silica), micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate. Certain additives for use with the present invention, and include: Talc: Talc is a purified, hydrated, magnesium silicate. It is widely used in oral solid dosage forms as a lubricant and diluent.

In some compositions, additives may also include: chelating agents (such as EDTA and EDTA salts); colorants or opaquants (such as titanium dioxide, food dyes, lakes, natural vegetable colorants, iron oxides, silicates, sulfates, magnesium hydroxide and aluminum hydroxide); coolants (e.g., trichloroethane, trichloroethylene, dichloromethane, fluorotrichloromethane); cryoprotectants (such as trehelose, phosphates, citric acid, tartaric acid, gelatin, dextran and mannitol); and diluents or fillers (such as lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose). Yet other additives may include disintegrants or super disintegrants; hydrogen bonding agents, such as magnesium oxide; flavorants or desensitizers.

It should be appreciated that there is considerable overlap between the above-listed additives and/or active agent in common usage, since a given additive/active agent is often classified differently by different practitioners in the field, or is commonly used for any of several different functions. Thus, the above-listed additives should be taken as merely exemplary, and not limiting, of the types of additives that can be included in compositions of the present invention. The amounts of such additives may be readily determined by one skilled in the art, according to the particular properties desired.

A pelletization process typically involves preparing a molten solution of the composition of the solid carrier or a dispersion of the composition of the solid carrier solubilized or suspended in an aqueous medium, an organic solvent, a supercritical fluid, or a mixture thereof. Such solution or dispersion is then passed through a certain opening to achieve the desired shape, size, and other properties. Similarly, appropriate drying processes may be used to control the level of the residual dispersing medium, if necessary. The processes described above, the combination of the processes, or the modifications of the processes are known in the art. Some of the processes are briefly described herein for reference.

In a broad sense, pellets are very much like granules and bead; the techniques for producing pellets may also produce granules, beads, etc. Pellets, granules or beads are formed with the aid of, e.g., a pelletizer, a spheronizer or an extruder. The pelletizer, spheronizer or extruder is able to form approximately spherical bodies from a mass of finely divided particles continuously, by a rolling or tumbling action on a flat or curved surface with the addition of a liquid.

Pelletizers are generally classified based on the angle of their axis as a horizontal drum or an inclined dish pelletizer. Rotary fluidized granulators may also be used for pelletization. A standard fluidized drier bowl may be replaced with a rotating plate as an air distributor. For granulation, a binder liquid is sprayed from via one or two binary nozzles located axially to the rotational movement of the powder bed. The granulation results in rounding of the granules to approximately spherical pellets. Such balling or agitation techniques are generally influenced by operating conditions, e.g., the bridging/binding liquid requirements, the residence time of the material in the pelletizer, the speed and angle of inclination of the pelletizer, the amount of material fed to the pelletizer and the choice and levels of binder, etc. Those skilled in the art may adjust readily such factors to produce a satisfactory product.

A tablet may be made by compression or molding, optionally with one or more accessory ingredients. Compressed tablets may be prepared by compressing, in a suitable machine, the therapeutic ingredient in a free-flowing form such as a powder or granules, optionally mixed with a binder, lubricant, inert diluent, preservative, surface therapeutic or dispersing agent. Molded tablets may be made by molding, in a suitable machine, a mixture of the powdered compound moistened with an inert liquid diluent. The tablets may be optionally coated or scored and may be formulated so as to provide a slow or controlled release of the therapeutic ingredient therein.

The choice of binder for a given application may also be determined readily by those skilled in the art. Generally, the binder must be capable of wetting the surfaces of the particle being pelletized or granulated. In general, binders must have sufficient wet strength to allow agglomerates to be handled and sufficient dry strength to make them suitable for their intended purposes. Each process, however, makes use of a different system of forces and may require a different agglomerate strength. The final selection of the binder is made generally based on the type of equipment used. Factors that affect the equipment and binder choices include: the size and size distribution of pellets, bulk density, strength and flow properties. Other factors that affect the performance of the pellets, which may be adjusted by one skilled in the art by the inclusion of additives, choice of equipment and processing conditions.

For example, suitable polymers for use with the present invention include, but are not limited to, synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll®, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof.

The blend of polymers may form a hydrogel or matrix using a material such as a carbohydrate polymer or polysaccharide (e.g., hyaluronic acid) in the presence of an initiator such as mono-, di- or trivalent cations or anions in water, a radical, or a photoinitiator. The polymer blend may be intrinsically biodegradable, biocompatible, or of sufficiently low molecular weight to allow excretion. Some components of the polymer blend exhibit little to no ability to biologically degrade. Where there are two or more water-soluble polymer blocks joined by other groups, the joining groups may include biodegradable linkages, polymerizable linkages, or both.

Other polymer formulations for use with the present invention include scaffolds prepared with the polymer of the present invention and one or more bioactive compounds or active species so that the polymer or scaffold becomes a microcarrier for one or more active species. The active species may be incorporated into the polymer or polymer solution (e.g., scaffold) or may be attached to its surface using techniques readily apparent to those skilled in the art. In some instances, it may be preferred to incorporate or attach a precursor of the active agent, e.g., an inactive version of the species that can then be activated to the active species as needed and required. The active species may be a drug or other biologically active compound; thus, the scaffold may be a microcarrier for the delivery of drugs or other biologically active compounds when used in the body. Examples of biologically active compounds are proteins, peptides, polysaccharides, nucleic acids, oligonucleotides, natural and synthetic organic or inorganic molecules, and those biologic molecules used for therapeutic, prophylactic or diagnostic purposes. Drugs may include antibiotics, antivirals, chemotherapeutic agents, anti-angiogenic agents, hormones, anti-inflammatory agents, drugs having an effect on vascular flow or that are effective against one or more diseases, and combinations thereof.

Active agents (excipients) of the present invention can also include decongestants (along with a salt form). Examples include, but are not limited to, phenylephrine (bitartrate, tannate, HBr, HCl), phenylpropanolamine (HCl) and pseudoephedrine (HCl). Furthermore, a number of herbal and/or natural decongestants are known in the art, all of which can be used with the present invention.

Active agents such as expectorants can also be used with the present invention. e.g., guaifenesin, terpin hydrate, (glyceryl guaiacolate), potassium (iodide, citrate) and potassium guaicolsulfonate. Other expectorants, whether individual ingredients or combinations of ingredients may be used with the present invention. Furthermore, a number of herbal and/or natural expectorants are known in the art, all of which may be used with the present invention, e.g., Oregano Leaf Extract 25-500 mg (which may be a liquid extract), Red Clover 25-500 mg, Buckthorn Root 25-500 mg, or Fenugreek 25-500 mg, or mixtures thereof.

Examples of antihistamines for use as active agents with the present invention (e.g., in salt form) are chlorpheniramine (maleate), brompheniramine (maleate), dexchlorpheniramine (maleate), dexbrompheniramine (maleate), triprolidine (HCl), diphenhydramine (HCl), doxylamine (succinate), tripelennamine (HCl), cyproheptatine (HCl), bromodiphenhydramine (HCl), phenindamine (tartrate), pyrilamine (maleate, tannate) and azatadine (maleate). Antitussives that may be used with the present invention (with salt form) include: caramiphen (edisylate), dextromethorphan (HBr) and codeine (phosphate, sulfate). A number of herbal and/or natural antihistamines are known in the art, all of which may be used with the present invention.

Other actives may also be included with the present invention, e.g., non-steroidal anti-inflammatory drugs (NSAIDs) such as propionic acid derivatives; acetic acid derivatives; fenamic acid derivatives; biphenylcarboxylic acid derivatives; and oxicams. Examples of propionic acid derivatives include: ibuprofen, naproxen, ketoprofen, flurbiprofen, fenoprofen, suprofen, fenbufen, and fluprofen may be mentioned as preferred compounds. Acetic acid derivatives include: tolmetin sodium, zomepirac, sulindac and indomethacin. Fenamic acid derivatives include: mefenamic acid and meclofenamate sodium. Diflunisal and flufenisal are biphenylcarboxylic acid derivatives, while oxicams include piroxicam, sudoxicam and isoxicam. Other analgesics for use with the present invention include acetaminophen and phenacetin.

In some embodiments, the present invention can include pharmaceutical Glaze (e.g., Shellac). Shellac is a natural occurring material, consisting of a complex mixture of constituents. The main component of shellac (˜95%) is a resin that upon mild basic hydrolysis gives a mixture of compounds of high plasticity. Shellac is used extensively in the pharmaceutical industry as a film coating agent for beads and tablets.

The one or more active agents that are formulated in a self-stable manner using the present invention may include a wide variety of uses, not just the traditional pharmaceutical agents. Actives for use with the present invention in immediate and/or controlled release formulations may include systemically active therapeutic agents, locally active therapeutic agents, disinfecting agents, chemical impregnants, cleansing agents, deodorants, fragrances, dyes, animal repellents, insect repellents, fertilizing agents, pesticides, herbicides, fungicides, and plant growth stimulants, and the like. Although some examples of active agents are listed, those skilled in the art will appreciate that any of these compounds may be used in the form of their pharmaceutically acceptable salt forms, e.g., carboxylic acids, with counter-ions, e.g., potassium, sodium, calcium; as ionic combinations with, e.g., resins, polymers, beads, matrices; with sugars or sugar derivatives, e.g., malate, tannate; amino acids, lipids, oils or combinations, mixtures and the like. In some embodiments, the present inventors have found that certain actives may be provided with two different salts, each of which may have a different solubility and/or release profile under, e.g., physiologic conditions.

Some other examples of active agents (ingredients) suitable for use in the pharmaceutical formulations and methods of the present invention include: hydrophilic, lipophilic, amphiphilic or hydrophobic, and that can be solubilized, dispersed, or partially solubilized and dispersed, on or about a carrier. The active agent-carrier combination may be coated further to encapsulate the agent-carrier combination. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmaceuticals, nutraceuticals, diagnostic agents, nutritional agents, and the like. The active agents listed below may be found in their native state, however, they will generally be provided in the form of a salt. The active agents listed below include their isomers, analogs and derivatives.

In one embodiment, the active ingredient agent is hydrophobic. Hydrophobic active ingredients are compounds with little or no water solubility. Intrinsic water solubilities (i.e., water solubility of the unionized form) for hydrophobic active ingredients are less than about 1% by weight, and typically less than about 0.1% or 0.01% by weight. Suitable hydrophobic active ingredients are not limited by therapeutic category, and can be, for example, analgesics, anti-inflammatory agents, antihelmimthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastrointestinal agents, histamine receptor antagonists, keratolytics, lipid regulating agents, anti-anginal agents, cox-2 inhibitors, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof. Salts, isomers and derivatives of the above-listed hydrophobic active ingredients may also be used, as well as combinations and mixtures thereof.

Other examples of suitable hydrophobic active ingredients include: acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, and zopiclone. Of course, salts, isomers and derivatives of the above-listed hydrophobic active ingredients may also be used, as well combinations and mixtures thereof.

In other embodiments, the active ingredient is hydrophilic, however, combination of hydrophilic, hydrophobic and non-polar agents may also be used. The water solubility for hydrophilic active ingredients is generally greater than about 0.1% by weight, and typically greater than about 1% by weight. Suitable hydrophilic active ingredients include: analgesics, anti-inflammatory agents, antihelmimthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, beta-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof

Other hydrophilic active ingredients include: a cytokine, a peptidomimetic, a peptide, a protein, a toxoid, a serum, an antibody, a vaccine, a nucleoside, a nucleotide, a portion of genetic material, a nucleic acid, or a mixture thereof. Other examples of suitable hydrophilic active ingredients include: acarbose; acyclovir; acetyl cysteine; acetylcholine chloride; alatrofloxacin; alendronate; aglucerase; amantadine hydrochloride; ambenomium; amifostine; amiloride hydrochloride; aminocaproic acid; amphotericin B; antihemophilic factor (human), antihemophilic factor (porcine); antihemophilic factor (recombinant), aprotinin; asparaginase; atenolol; atracurium besylate; atropine; azithromycin; aztreonam; BCG vaccine; bacitracin; becalermin; belladonna; bepridil hydrochloride; bleomycin sulfate; calcitonin human; calcitonin salmon; carboplatin; carbohydrate and carbohydrate polymers, capecitabine; capreomycin sulfate; cefamandole nafate; cefazolin sodium; cefepime hydrochloride; cefixime; cefonicid sodium; cefoperazone; cefotetan disodium; cefotaxime; cefoxitin sodium; ceftizoxime; ceftriaxone; cefuroxime axetil; cephalexin; cephapirin sodium; cholera vaccine; chorionic gonadotropin; cidofovir; cisplatin; cladribine; clidinium bromide; clindamycin and clindamycin derivatives; ciprofloxacin; clodronate; colistimethate sodium; colistin sulfate; corticotropin; cosyntropin; cromolyn sodium; cytarabine; dalteparin sodium; danaparoid; desferrioxamine; denileukin diflitox; desmopressin; diatrizoate meglumine and diatrizoate sodium; dicyclomine; didanosine; dirithromycin; dopamine hydrochloride; dornase alpha; doxacurium chloride; doxorubicin; etidronate disodium; enalaprilat; enkephalin; enoxaparin; enoxaprin sodium; ephedrine; epinephrine; epoetin alpha; erythromycin; esmolol hydrochloride; factor IX; famciclovir; fludarabine; fluoxetine; foscarnet sodium; ganciclovir; granulocyte colony stimulating factor, granulocyte-macrophage stimulating factor; growth hormones-recombinant human; growth hormone-bovine; gentamycin; glucagon; glycopyrolate; gonadotropin releasing hormone and synthetic analogs thereof. GnRH; gonadorelin; grepafloxacin; haemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human, insulin lispro; insulin procine; insulin NPH; insulin aspart; insulin glargine; insulin detemir; interferon alpha; interferon beta; ipratropium bromide; ifosfamide; Japanese encephalitis virus vaccine; lamivudine; leucovorin calcium; leuprolide acetate, levofloxacin; lincomycin and lincomycin derivatives; lobucavir; lomefloxacin; loracarbef; mannitol; is measles virus vaccine; meningococcal vaccine; menotropins; mepenzolate bromide; mesalamine; methenamine; methotrexate; methscopolamine; metformin hydrochloride; metoprolol; mezocillin sodium; mivacurium chloride; mumps viral vaccine; nedocromil sodium; neostigmine bromide; neostigmine methyl sulfate; neurontin; norfloxacin; octreotide acetate; ofloxacin; olpadronate; oxytocin; pamidronate disodium; pancuronium bromide; paroxetine; perfloxacin; pentamidine isethionate; pentostatin; pentoxifylline; periciclovir; pentagastrin; pentholamine mesylate; phenylalanine; physostigmine salicylate; plague vaccine; piperacillin sodium; platelet derived growth factor-human; pneumococcal vaccine polyvalent; poliovirus vaccine inactivated; poliovirus vaccine live (OPV); polymyxin B sulfate; pralidoxime chloride; pramlintide, pregabalin; propafenone; propenthaline bromide; pyridostigmine bromide; rabies vaccine; residronate; ribavarin; rimantadine hydrochloride; rotavirus vaccine; salmeterol xinafoate; sinealide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; tumor necrosis factor; typhoid vaccine live; urea; urokinase; vancomycin; valacyclovir; valsartan; varicella virus vaccine live; vasopressin and vasopressin derivatives; vecuronium bromide; vinblastine; vincristine; vinorelbine; vitamin B12; warfarin sodium; yellow fever vaccine; zalcitabine; zanamivir; zolendronate; zidovudine; pharmaceutically acceptable salts, isomers and derivatives thereof; and mixtures thereof.

A wide variety of therapeutically active agents can be used in conjunction with the present invention. The therapeutically active agents (e.g. pharmaceutical agents) which may be used in the compositions of the present invention include both water soluble and water insoluble drugs. Examples of such therapeutically active agents include antihistamines (e.g., dimenhydrinate, diphenhydramine, chlorpheniramine and dexchlorpheniramine maleate), analgesics (e.g., aspirin, codeine, morphine, dihydromorphone, oxycodone, etc.), non-steroidal anti-inflammatory agents (e.g., naproxyn, diclofenac, indomethacin, ibuprofen, sulindac), anti-emetics (e.g., metoclopramide), anti-epileptics (e.g., phenyloin, meprobamate and nitrezepam), vasodilators (e.g., nifedipine, papaverine, diltiazem and nicardirine), anti-tussive agents and expectorants (e.g., codeine phosphate), anti-asthmatics (e.g. theophylline), antacids, anti-spasmodics (e.g., atropine, scopolamine), antidiabetics (e.g., insulin), diuretics (e.g., ethacrynic acid, bendrofluazide), anti-thypotensives (e.g., propranolol, clonidine), antihypertensives (e.g, clonidine, methyldopa), bronchodilators (e.g., albuterol), steroids (e.g., hydrocortisone, triamcinolone, prednisone), antibiotics (e.g., tetracycline), antihemorrhoidals, hypnotics, psycho-tropics, antidiarrheals, mucolytics, sedatives, decongestants, laxatives, vitamins, stimulants (including appetite suppressants such as phenylpropanolamine), as well as salts, hydrates, and solvates of the same. The above list is not meant to be exclusive.

In certain embodiments, the therapeutically active agent comprises hydromorphone, oxycodone, dihydrocodeine, codeine, dihydromorphine, morphine, buprenorphine, salts, hydrates and solvates of any of the foregoing, mixtures of any of the foregoing, and the like. In other embodiments, the active agent is a locally active therapeutic agent and the environment of use may be, e.g., the gastrointestinal tract, or body cavities such as the oral cavity, periodontal pockets, surgical wounds, the rectum or vagina. The liquid formulations of the present invention may be provided orally, topically, subcutaneously, intramuscularly, intraperitoneally, intraocularly, intraossealy, nasally, urethrally, mucosally, vaginally, rectally, intradurally, epidurally and the like. The liquid formulation of the present invention may also be provided as a mist, e.g., to the deep lung (alveolarly).

Locally active pharmaceutical agents of use with the present invention include antifungal agents (e.g., amphotericin B, clotrimazole, nystatin, ketoconazole, miconazol, etc.), antibiotic agents (penicillins, cephalosporins, erythromycin, tetracycline, aminoglycosides, etc.), antiviral agents (e.g., acyclovir, idoxuridine, etc.), breath fresheners (e.g. chlorophyll), antitussive agents (e.g., dextromethorphan hydrochloride), anti-cariogenic compounds (e.g. metallic salts of fluoride, sodium monofluorophosphate, stannous fluoride, amine fluorides), analgesic agents (e.g., methylsalicylate, salicylic acid, etc.), local anesthetics (e.g., benzocaine), oral anti-septics (e.g., chlorhexidine and salts thereof, hexylresorcinol, dequalinium chloride, cetylpyridinium chloride), anti-inflammatory agents (e.g., dexamethasone, betamethasone, prednisone, prednisolone, triamcinolone, hydrocortisone, etc.), hormonal agents (oestriol), antiplaque agents (e.g, chlorhexidine and salts thereof, octenidine, and mixtures of thymol, menthol, methysalicylate, eucalyptol), acidity reducing agents (e.g., buffering agents such as potassium phosphate dibasic, calcium carbonate, sodium bicarbonate, sodium and potassium hydroxide, etc.), and tooth desensitizers (e.g., potassium nitrate). This list is not meant to be exclusive. Other embodiments of the present invention include disinfecting agents, e.g., chlorine compounds such as calcium hypochlorite, and the environment of use is a surrounding body of water, e.g. a recreational pool. The active may be one or more cleansing agents, a germicide, a deodorant, a surfactant, a fragrance, a perfume, a sanitizer, and/or a dye, and the environment of use is an aqueous solution, e.g. a urinal or toilet bowl. Examples of fragrances include: perfume oils, volatile-compounds including esters, ethers aldehydes, alcohols, unsaturated hydrocarbons, terpenes, and other ingredients well known in the art.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A multilayer modular release system, composition comprising:
active agent release layers, wherein the layers form a stack of active agent release layers, wherein the stack comprises a body and first and second ends; and
an impermeable coating surrounding the body of the stack, wherein the active agent is only release from the first, second or both the first and second ends of the stack by diffusion.

2. The system of claim 1, wherein at least one of the active agent release layers comprises no active agent.

3. The system of claim 1, wherein the pharmaceutical composition comprise an extended release profile.

4. The system of claim 1, wherein the pharmaceutical composition comprise a pulsatile release profile.

5. The system of claim 1, wherein the pharmaceutical composition comprise a delayed release profile.

6. The system of claim 1, wherein the pharmaceutical composition comprise a rapid followed by a constant release profile.

7. The system of claim 1, wherein the pharmaceutical composition comprise an increasing release profile.

8. The system of claim 1, wherein the pharmaceutical composition comprise a concurrent release profile.

9. The system of claim 1, wherein the pharmaceutical composition comprises one or more release profiles selected from an extended release, a pulsatile, a delayed release, a rapid followed by a constant release, an increasing release and a concurrent release profile.

10. A method of making a multilayer modular release system comprising the steps of:
forming four or more active agent release layers, wherein the layers form a stack of active agent release layers, wherein the stack comprises a body and first and second ends; and
coating the stack with an impermeable coating that surrounds the body of the stack, wherein the active agent is only release from the first, second or both the first and second ends of the stack by diffusion.

11. The method of claim 10, wherein at least one of the active agent release layers comprises no active agent.

12. The method of claim 10, wherein the pharmaceutical composition comprise an extended release profile.

13. The method of claim 10, wherein the pharmaceutical composition comprise a pulsatile release profile.

14. The method of claim 10, wherein the pharmaceutical composition comprise a delayed release profile.

15. The method of claim 10, wherein the pharmaceutical composition comprise a rapid release followed by a constant release profile.

16. The method of claim 10, wherein the pharmaceutical composition comprise an increasing release profile.

17. The method of claim 10, wherein the pharmaceutical composition comprise a concurrent release profile.

18. The method of claim 10, wherein the pharmaceutical composition comprises one or more release profiles selected from an extended release, a pulsatile, a delayed release, a rapid release followed by a constant release, an increasing release and a concurrent release profile.

19. A multilayer, multiple profile drug release pharmaceutical composition comprising:
four or more discs that form a stack of discs, wherein each of the discs comprises an active agent releasing polymer and an active agent having a concentration of between 0 and 99.9% by weight of the disc, wherein the stack of discs comprises a body and first and second ends; and
an impermeable coating surrounding the body of the stack, wherein the active agent is only release from the first, second or both the first and second ends of the stack by diffusion.

20. The composition of claim 19, wherein each of the discs has the same concentration of active agent.

21. The composition of claim 19, wherein each of the adjacent discs has a different concentration of active agent.

22. The composition of claim 19, wherein one or more non-adjacent discs have the same concentration of active agent.

23. The composition of claim 19, wherein the pharmaceutical composition further comprises one or more layers of an extended-release coating.

24. The composition of claim 19, wherein the pharmaceutical composition further comprises one or more inactive agents.
